(19) United States
(12) Patent Application Publication
(10) Pub. No.: US 2017/0102374 A1
(43) Pub. Date: Apr. 13, 2017

(54) METHOD OF OBSERVING MICROORGANISM ACTIVITY IN A HUMAN TISSUE SAMPLE

(71) Applicant: Michael Earl Drabinsky, Walnut Creek, CA (US)

(72) Inventor: Michael Earl Drabinsky, Walnut Creek, CA (US)

(21) Appl. No.: 15/286,530

(22) Filed: Oct. 5, 2016

Related U.S. Application Data
(60) Provisional application No. 62/238,906, filed on Oct. 8, 2015.

(51) Int. Cl. G01N 33/483; G02B 21/00; G06K 9/00

(52) U.S. Cl. CPC G01N 33/4833; G06K 9/00134; G02B 21/0056; G02B 21/0028; G02B 21/0032

(57) ABSTRACT

A method of observing live microorganisms in an animal tissue sample over an extended duration of time which enables the detection and identification of multiple different microbes present in the sample is performed using a phase contrast microscope and begins with the preparation of a sample containing animal tissue for viewing with the microscope. Once the sample is prepared, an initial, baseline observation of the sample is performed using the microscope. Following the initial observation of the sample and after at least one selected time interval which allows objects in the sample to naturally undergo changes in morphology, at least one subsequent observation is performed with the microscope so as to analysis of the activity of selected objects in the sample. Through the analysis, microorganisms in the animal tissue may be detected and identified based on observed changes in morphology and biofilm characteristic.

### CROSS REFERENCE TO RELATED APPLICATIONS

This application claims the benefit of and incorporates by reference co-pending U.S. provisional patent application Ser. No. 62/238,906 filed Oct. 8, 2015.

### BACKGROUND OF THE INVENTION

Field of the Invention

The present invention relates generally to use of phase contrast light microscopy for detecting, identifying, or evaluating treatment progress of live microorganisms (or “microbes”) in human tissue samples on a slide.

Description of the Prior Art

Many health problems are caused by infections from bacteria, protozoa, or fungi, which are difficult to detect with a high level of accuracy. One of the most commonly used diagnosis methods for detecting or identifying pathogenic microorganisms (or “pathogens”), Serology, can detect antibodies related to a specific infectious agent, but only 1 at a time. Molecular diagnostics techniques generally use polymerase chain reaction (PCR) technology, but again, can only test for a specific organism at a time. Culture methods are used, but many organisms do not stay alive or grow with traditional culture plates, making this method largely ineffective. Light microscopy is not useful for many microbes because they are nearly transparent, and are therefore not visible enough. As a result it is difficult to diagnose and later evaluate treatment progression in an effective way.

Infections from tick bites, deer flies, mosquito bites, or other kinds of transmission are often characterized by multiple pathogens. This adds complexity to the treatment plan, since it becomes difficult to pinpoint various symptoms to a particular pathogen. Symptoms can persist or change after a course of treatment even after targeting a certain pathogen. It can be difficult to ascertain the cause of the symptoms, whether due to a different pathogen or insufficient treatment of the same targeted pathogen. Treatments often require multiple medications which further complicate the evaluation process. The presence of biofilm adds complexity in several ways. Biofilms provide a way for multiple pathogens to shield themselves from medications. Additionally, pathogens protected in biofilm can hide from the immune system and go undetected from traditional antibody testing methods.

There is not an effective system in use for evaluating with certainty what pathogens are causing various symptoms and therefore selecting the appropriate treatment. This is an ongoing problem with Lyme disease and associated infections. For example, a patient with both Lyme (Borrelia bacteria) and Babesia can be treated with drugs and find certain symptoms improved while other symptoms seem to have gotten worse. As such, it would be valuable to be able to look at the blood to see what is all there. Is Babesia present in significant numbers, or is there a prevalence of Lyme bacteria, Bartonella or other pathogen? Knowing this can guide the next step in treatment.

Biofilms are common in human and animal infections. A biofilm is comprised of microorganisms which secrete extracellular polymeric substances which form a matrix around groups of individual cells. The polymeric substances can change form and stick to each other or to blood vessel walls or any tissue nearby.

Biofilms can be observed in diverse forms and varied hues, colorations, shades or luminosities. Generally, it is understood that each micro-organism has its own unique biofilm structure. As such, each kind of microorganism can have a characteristic biofilm morphology, especially when observed as individual microorganisms, separate from clumps of biofilms. For example, biofilm that is characteristic of Babesia has a different morphology than biofilm of FL-1953 (a.k.a Protomyxzoa rheumatica). Other accumulations or clumps of biofilm when observed can appear larger or smaller, or as discrete shapes while others can appear more amorphous or irregular. Other samples might contain smaller fragments of biofilms. Observing biofilm before a particular drug regimen or treatment is used by the patient can be valuable when compared to a sample taken from the same patient after a period of treatment. For example, large chunks of biofilm can later appear as smaller yet more numerous fragments after a period of time, possibly indicating the treatment was effective at breaking up the biofilm clusters

When dealing with pathogens in biofilms, it can be very difficult to get antibiotics to diffuse through biofilm in an effective way to kill the various pathogens sequestered therein. Visual observation of pathogens and biofilm in blood or tissue has at times been used with cell staining microscopy, but with limited usefulness and poor acceptance by the medical community. Stains have been developed for various pathogens, but each stain only illuminates the particular pathogen it was designed for. If an unknown pathogen is present, it is not likely to be illuminated, and will therefore be missed. New stains must be invented for each newly identified microbe. A separate test must be run for each stain or pathogen selected. Selecting the right stain is critical to identification.

Phase contrast microscopy techniques provide an alternative which can allow one to differentiate between different types of molecules, microbes, biofilms, and so forth based upon differences in viewable color and morphology. In phase contrast microscopy, the difference in color is not due to actual colors of the organisms/biofilms themselves; but rather is due to the fact that different wavelengths of light travel at different speeds through more or less dense matter which phase contrast microscopy then separates and presents as different viewable colors. For example, a lipid micelle (drop of oil) will appear as a different shade of yellow than a Borrelia cyst, which is encapsulated in a lipophilic/saccharide matrix. This makes it possible to differentiate between potentially harmless fat drops in blood after eating an oil-rich meal, and, for example Borrelia cysts.

While phase contrast microscopy is well established, a problem which still exists is that phase contrast light microscopes have not generally been employed to observe the activity of live pathogens in order to identify and distinguish pathogens such as Borrelia, Babesia, and Protomyxzoa. Thus, there remains a need for a method of observing live microorganisms in animal tissue which allows for the observation of pathogens over a period of time so as to enable the identification and detection of multiple different microbes based on observed changes in morphology and biofilm characteristic as well as the monitoring of treatment protocols which have been employed.

### SUMMARY OF THE INVENTION

The present disclosure describes a method of observing live microorganisms in an animal tissue sample over an extended duration of time which enables the detection and identification of multiple different microbes present in the sample. The method of observing live microorganisms in an animal tissue sample over an extended duration of time is performed using a phase contrast microscope and begins with the preparation of a sample containing animal tissue for viewing with the microscope. Once the sample is prepared, an initial, baseline observation of the sample is performed using the microscope. Following the initial observation of the sample and after at least one selected time interval which allows objects in the sample to naturally undergo changes in morphology, at least one subsequent observation is performed with the microscope so as to analysis of the activity of selected objects in the sample. Through the analysis, which may include a comparison of the morphology of selected objects from the initial observation to the morphology of selected objects from subsequent observations, one or multiple specific microorganisms in the animal tissue may be detected and identified based on observed changes in morphology and biofilm characteristic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the steps of preparing a microbe culture for observation in accordance with the present invention.

FIG. 2 shows the steps of detecting live microorganisms in a microbe culture in accordance with the present invention.

FIG. 3 is a top plan view of a kit for obtaining a tissue sample for culturing and observing and a microscope for viewing the same in accordance with the present invention.

FIG. 4 shows an exemplary microbe culture image with live microorganisms from a phase contrast microscope in accordance with the present invention as Borrelia in spirochete form from blood sample in a sample that was cultured then dried.

FIG. 5 shows an exemplary microbe culture image with live microorganisms from a phase contrast microscope in accordance with the present invention as Borrelia medusa forms after using the instant slide culturing method in a sample taken from saliva.

FIG. 6 shows an exemplary microbe culture image with live microorganisms from a phase contrast microscope in accordance with the present invention as Borrelia spirochete coming out of cyst.

FIG. 7 shows an exemplary microbe culture image with live microorganisms from a phase contrast microscope in accordance with the present invention as free floating, motile, protozoa in a fresh blood sample.

FIG. 8 shows an exemplary microbe culture image with live microorganisms from a phase contrast microscope in accordance with the present invention as a sample of blood with lots of Babesia therein, with a plurality of red blood cells being shown and each mark inside the cells being a Babesia.

FIG. 9 shows an exemplary microbe culture image with live microorganisms from a phase contrast microscope in accordance with the present invention as FL1953 (Protomyxzoa) biofilm columns taken using phase contrast.

FIG. 10 shows an exemplary microbe culture image with live microorganisms from a phase contrast microscope in accordance with the present invention as FL1953 (Protomyxzoa) biofilm columns taken using phase contrast.

FIG. 11 shows an exemplary microbe culture image with live microorganisms from a phase contrast microscope in accordance with the present invention as a FL1953 (Protomyxzoa) biofilm column in greater detail that shows various microbes in the column.

FIG. 12 shows an exemplary microbe culture image with live microorganisms from a phase contrast microscope in accordance with the present invention as a FL1953 (Protomyxzoa) biofilm sheet in blood.

FIG. 13 shows an exemplary microbe culture image with live microorganisms from a phase contrast microscope in accordance with the present invention as a FL1953 biofilm sheet in blood.

FIG. 14 shows an exemplary microbe culture image with live microorganisms from a phase contrast microscope in accordance with the present invention.

FIG. 15 shows an exemplary microbe culture image with live microorganisms from a phase contrast microscope in accordance with the present invention with the white dots inside and outside the cell being mostly Babesia.

FIG. 16 shows an exemplary microbe culture image with live microorganisms from a phase contrast microscope in accordance with the present invention showing Borrelia as white lines.

FIG. 17 shows an exemplary microbe culture image with live microorganisms from a phase contrast microscope in accordance with the present invention as Lyme and Lyme biofilm after being cultured in accordance with the present invention.

FIG. 18 shows an exemplary microbe culture image with live microorganisms from a phase contrast microscope in accordance with the present invention as Borrelia with a cyst.

FIG. 19 shows an exemplary microbe culture image with live microorganisms from a phase contrast microscope in accordance with the present invention.

FIG. 20 shows an exemplary microbe culture image with live microorganisms from a phase contrast microscope in accordance with the present invention.

### DETAILED DESCRIPTION OF THE INVENTION

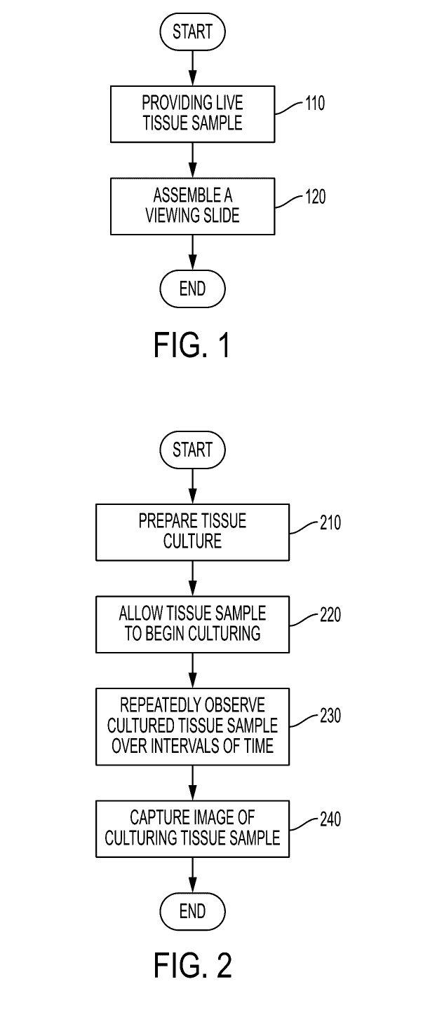

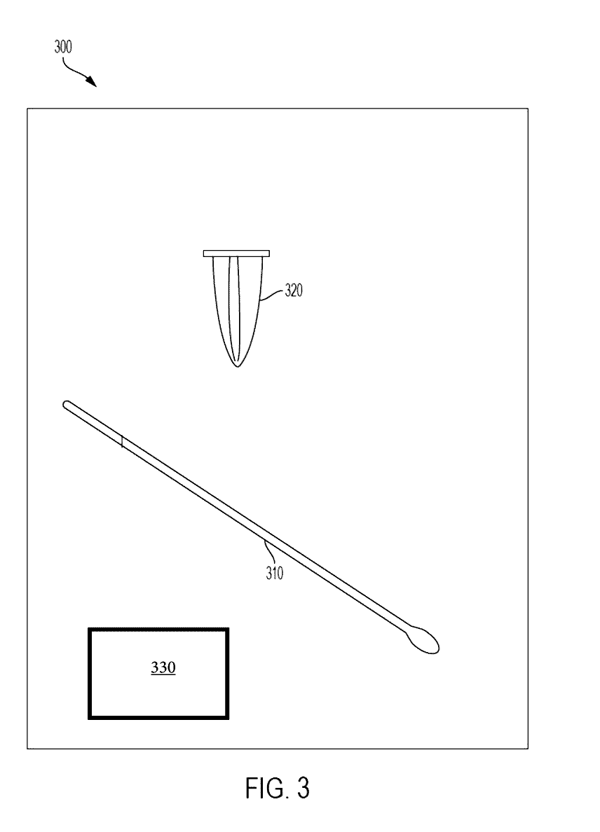

Referring now to the drawings and in particular FIGS. 1, 2, and 3, a method of observing live microorganisms in a human tissue sample is based on the preparation of a microbe culture for viewing under a specifically configured phase contrast microscope and the observation of the microbe culture over a period of time with the specifically configured phase contrast microscope. The process of preparing a microbe culture begins with the collection of a live tissue sample from a patient using a collection kit 300 which includes a cheek swab 310 and a collection vial 320. In the preferred embodiment, the live tissue sample may define a saliva sample and may be collected using the cheek swab 310, defined in one embodiment as a wad of cotton on a small stick. In such an embodiment, the swab 310 is brushed inside the cheek of a patient and then put in the collection vial 320 containing pure water. While in the vial 320, the swab 310 should be gently agitated to dislodge saliva containing various cells from the swab into the water. The swab 310 is then removed from the vial 320 and may be discarded. The vial 320, containing a saliva mix (namely, the saliva dislodged from the swab 310 and the pure water which was in the vial 320) is then provided for microscopy 110.

It is contemplated that, while the sample may be collected at the same location that has the requisite microscopy equipment, the collection kit 300 may alternatively be provided for tissue collection in a location other than a location having a microscopy lab. In such a scenario, the kit 300 could be sent to (or otherwise provided to) individuals who would then swab their own cheeks (or other tissues or blood) and cap and return the vial 320 with their tissue to a lab having the requisite microscopy equipment, allowing their sample to be cultured in accordance with the present invention.

Advantageously, the instant process does not use any genetic components. Moreover, it is noted that the vial 320 does not contain any growth media.

Once the tissue sample is collected, the process of preparing a microbe culture in the tissue sample continues with assembly of a viewing slide 120. To assemble the viewing slide, a small amount of the saliva mix is first placed on a conventional glass microscope slide. In one embodiment, this is accomplished by drawing up from the vial 320 a small amount of saliva mix into a pipette and placing a single drop on the slide. A glass cover slip is then gently positioned on the glass microscope slide, directly on top of the droplet of saliva mix and allowed to rest there, with no additional pressure or movement. At this point, the droplet of saliva mix defines the medium to support and culture the live microorganism and the tissue sample is prepared 210.

It is noted that traditional methods of fixing a slide for viewing usually kill or substantially alter the microbes. The preparation of a tissue sample in accordance with the above method, however, does not.

In one embodiment, the first observation of the tissue sample would take place within approximately 30 minutes after the viewing slide is first assembled. After the first observation, the tissue sample is then repeatedly observed again over an extended period of time at selected intervals of time 220 as discussed in greater detail below.

The observation would be performed with a phase contrast microscope 330 which has been specifically configured with a light emitting diode (“LED”) lamp in place of a tungsten-halogen lamp (or other halogen lamps), fluorescent bulb, or other lighting element. In addition, it is desirable for the magnification of the microscope to be set to between 400×-2000× and for all the appropriate phase contrast settings to be checked and adjusted as needed, so the microbes are fully in focus and sharp. In one embodiment, the specifically configured phase contrast microscope must have an ability to resolve features 400 nm or smaller. In accordance with the same, it is desirable for the specifically configured phase contrast microscope to have, at minimum, the ability to resolve features below roughly 350 nm.

It is appreciated that at present time, the phase contrast microscopy technology may be the only non-ribonucleic acid technology that can see biofilms. Indeed, most biofilms don't show up when using anything other than phase contrast or a nucleic acid probe (fluorescence).

It is contemplated that in practicing the instant invention, a microscope having an Infinity Corrected Optical System that includes Semi Plan Apochromatic Phase Contrast objective lenses would be desirable. Plan Apochromatic technology lenses, which are similar to semi-plan Apochromatic lenses, may also be sufficient. It would also be desirable to have a Zernike condenser with adjustable phase settings matched to each corresponding objective lens. In this regard, a Meiji Techno MT5210/5310 Laboratory Phase Contrast Microscope that includes 20× eyepieces (instead of 10×), U Plan semi-apochromatic, infinity corrected objective lenses ranging from 10×-100×, a Zernike phase condenser with adjustable phase positions matched to each objective lens, a centering telescope, and an LED for illumination would generally be sufficient to view a microbe culture prepared in accordance with the present invention. It is contemplated that a microscope configured in such a fashion would be configured to resolve features at 200×-2000× and below 350 nm in a phase contrast setting. For example, it is appreciated that a microscope in accordance with the present invention may be employed with 20× eyepieces and 100× objective lens which are phase enabled in order to provide 2000× magnification while still providing the requisite resolution.

It is appreciated, however, that it may be desirable to employ a microscope which includes phase enabled objective lenses which enable it to resolve features at 400×-4000×.

It is appreciated that though many microbes are nearly transparent, phase contrast makes them visible and defines various details. Significantly, the LED lamp supplies the requisite illumination without producing a significant amount of heat, thereby maintaining the microbe culture at a hospitable temperature close to a room temperature and allowing the microorganism in the culture to remain alive.

With the tissue culture prepared, the next step in the method of observing live microorganisms in a human tissue sample is to allow the tissue sample to begin culturing 220. As the tissue sample cultures over time, the sample is repeatedly observed 230 over an extended period of time at selected intervals of time. Because the microorganisms and cells in the tissue sample remain alive, this step allows for the identification, detection and viewing of a microorganism in human tissue over an extended period of time as they culture naturally through changes in morphology and biofilm characteristics. In the event that pathogens are observed, this method also enables the evaluation of treatment progress relating to the live pathogens on a slide based on the observed activity of the pathogens.

Advantageously, the extended time over which the microorganisms may be observed in an analog of their environment allow for observations of their formation and interaction with biofilms, cells, and other objects in the tissue sample. Indeed, it is contemplated that when observed in accordance with the present invention, microorganisms on the viewing slide can stay alive and continue to be observed at regular intervals for extended periods of time, possibly up to several days. It is understood, however, that under such a circumstance, phase contrast clarity would desirably be checked regularly or at least when objects appear blurred and cannot be put into focus—allowing for appropriate adjustments to be made so focus can remain dialed in clearly.

In addition, it is contemplated that for recording, documentation, and/or analysis purposes, it may be desirable to capture images 240 of the tissue sample being viewed. In one embodiment of the method of observing live microorganisms in a human tissue sample, a discrete camera may be employed to capture still or video images through the eyepiece len(s) of the microscope through which the tissue sample is being viewed. In such an embodiment, it is contemplated that the camera lens on the discrete camera capturing the still or video images is desirably sized with a diameter of less than 7 millimeters or otherwise less than the diameter of the eyepiece lens of the microscope through which the tissue sample is being viewed.

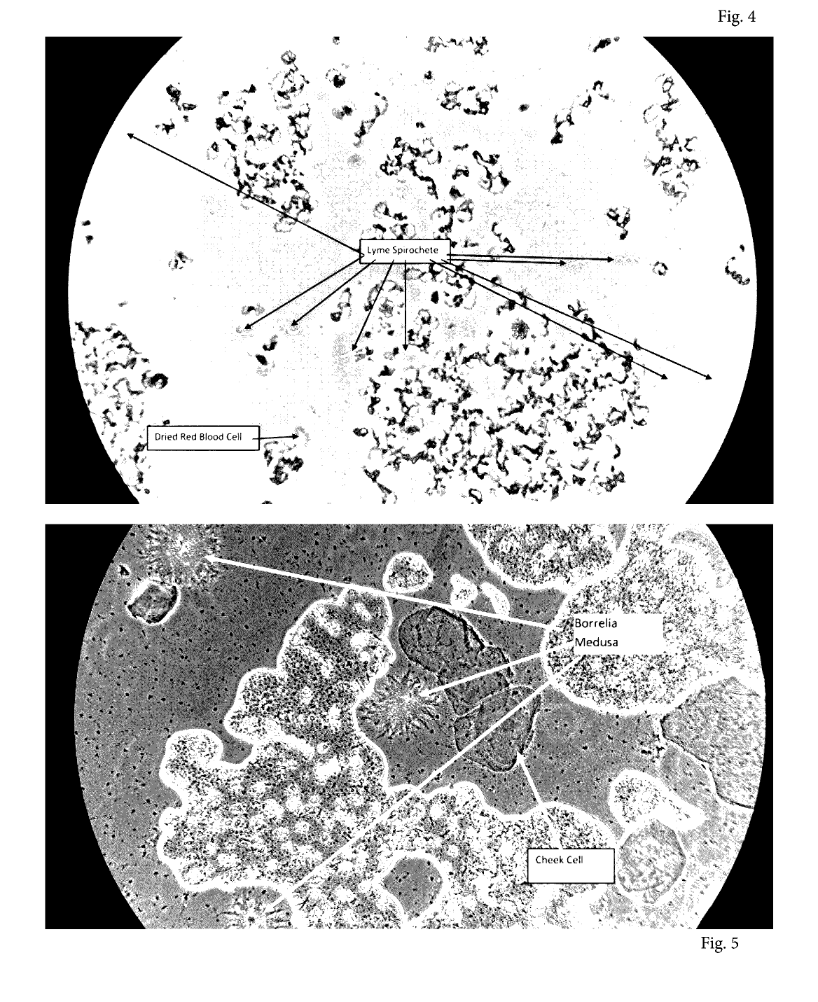

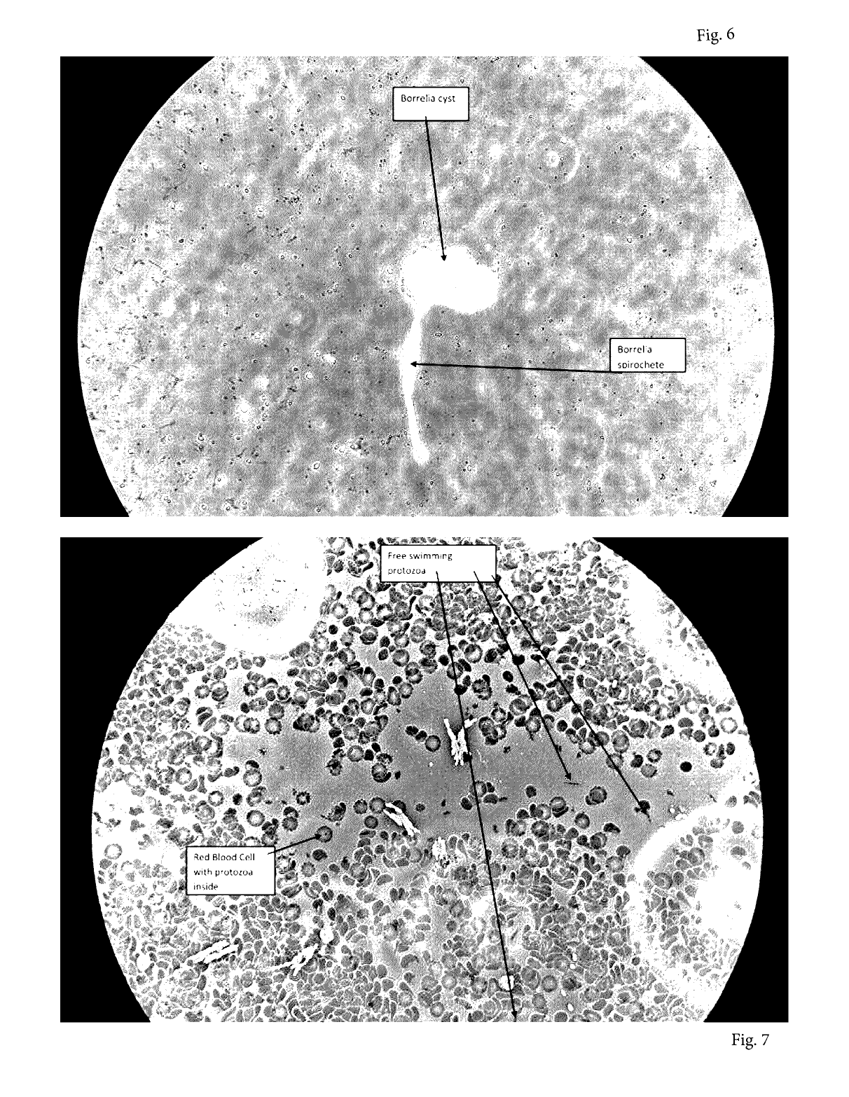

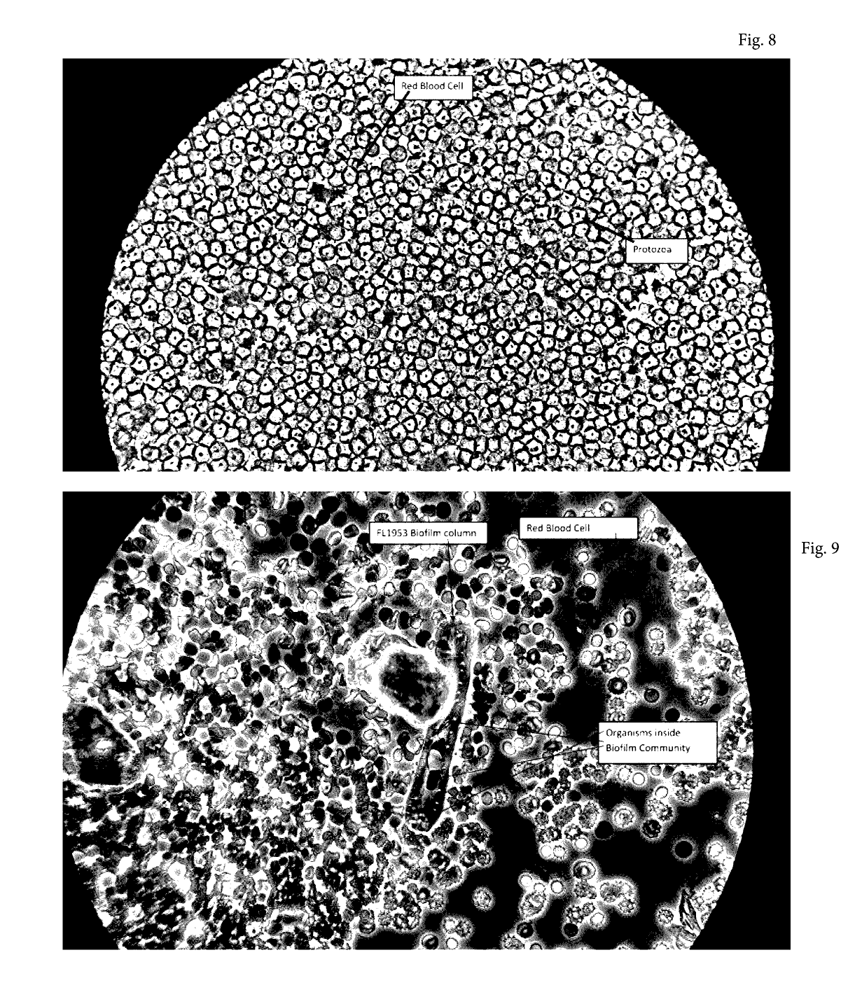

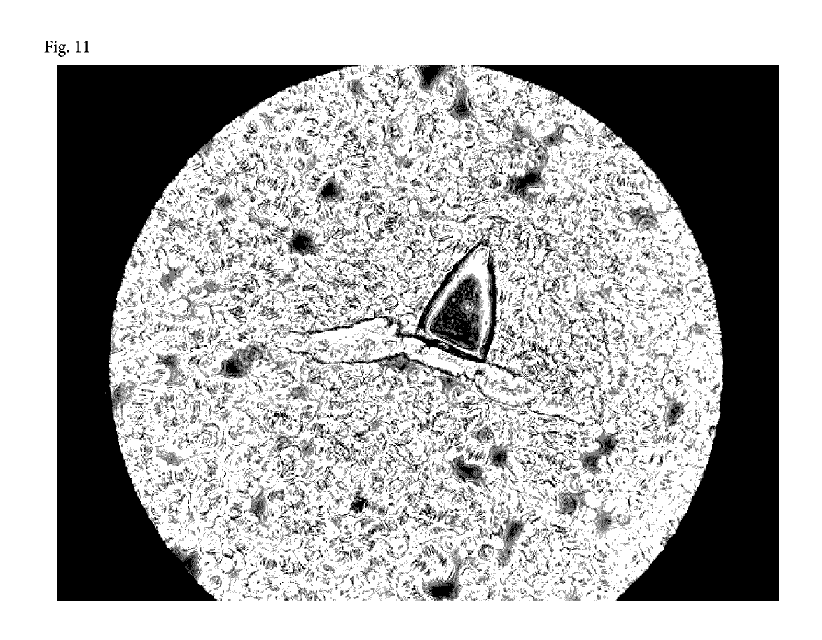

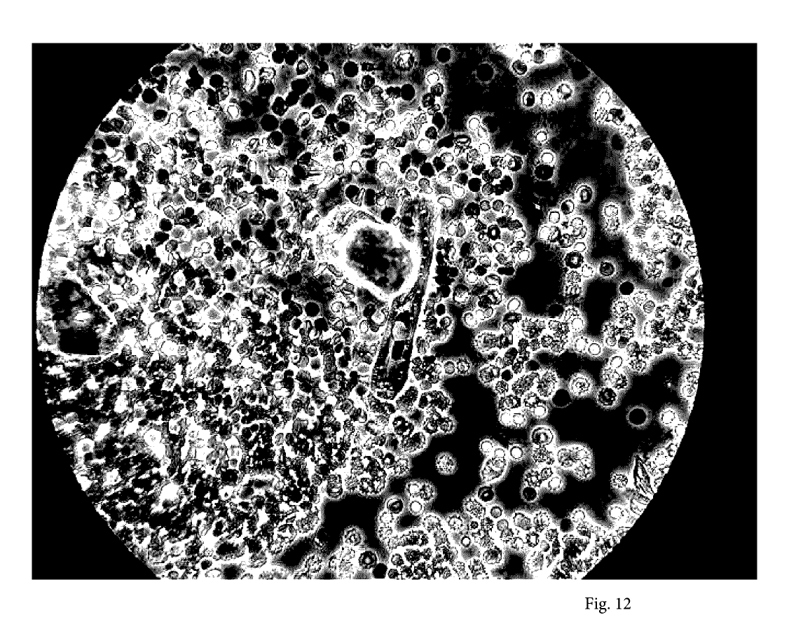

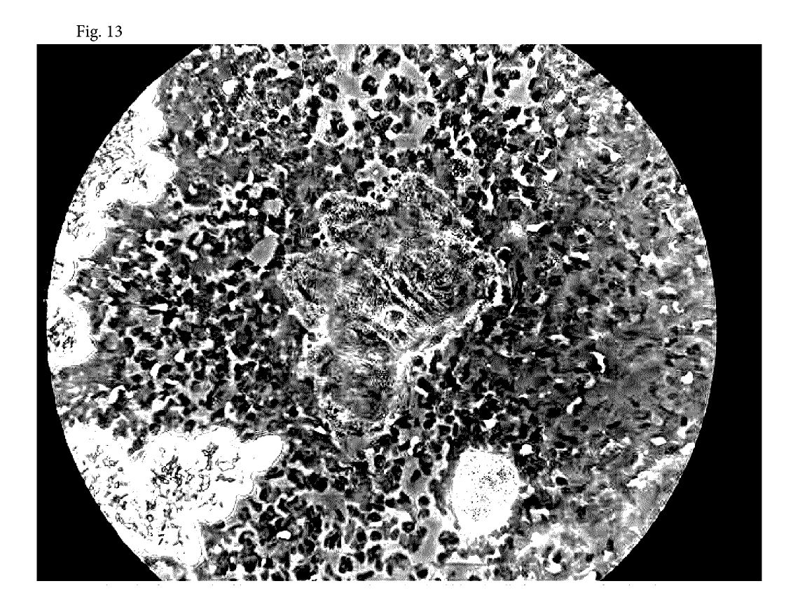

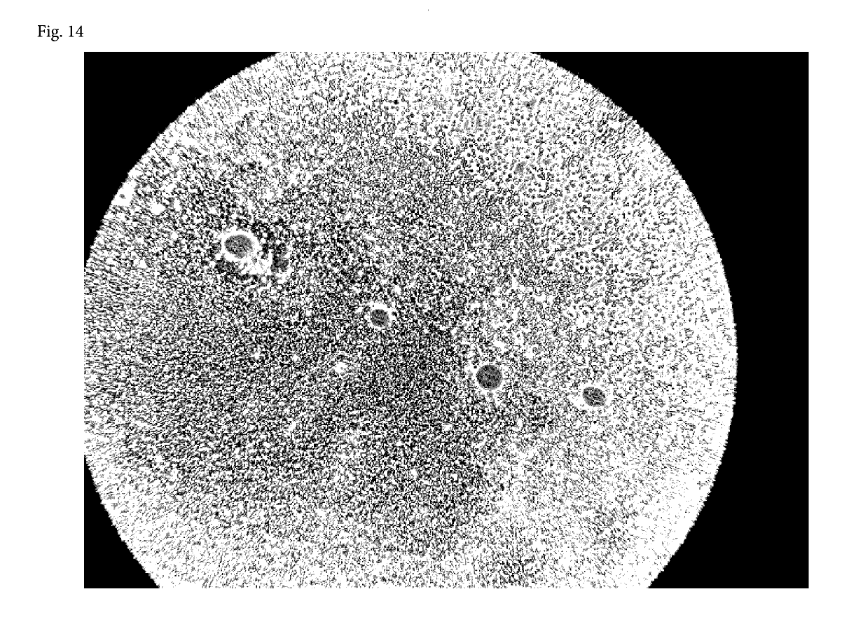

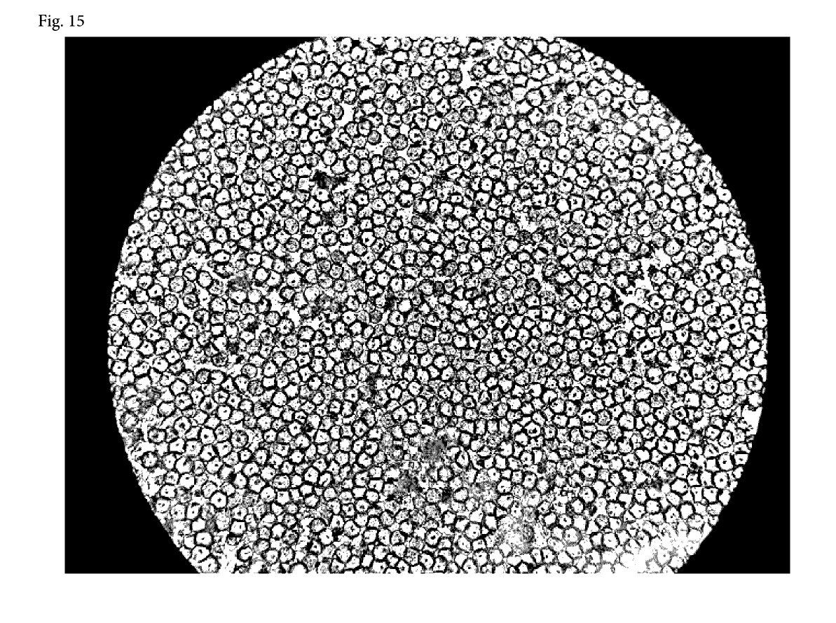

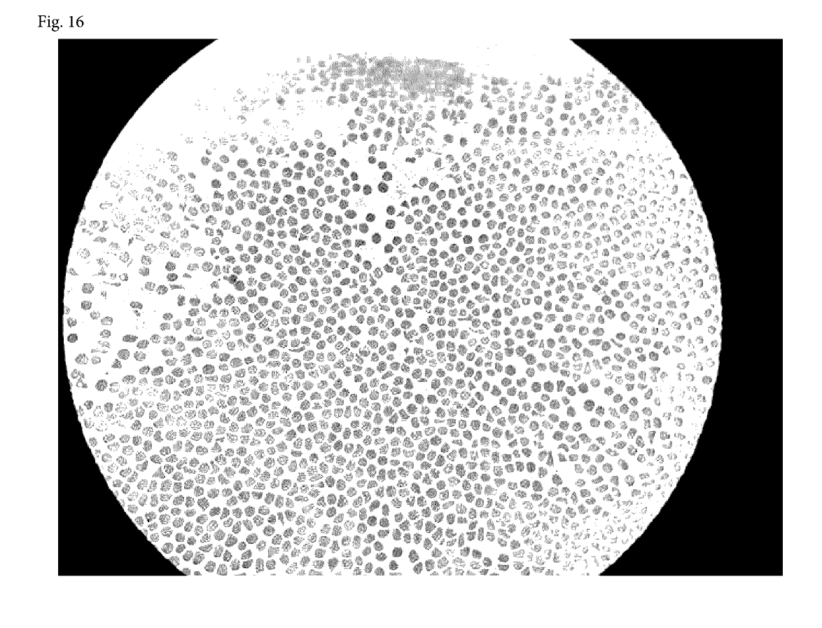

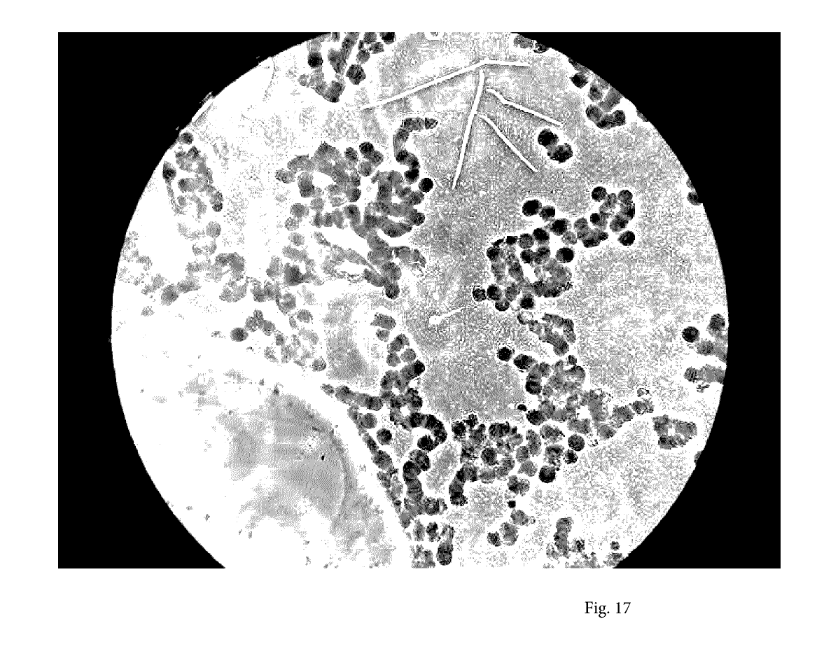

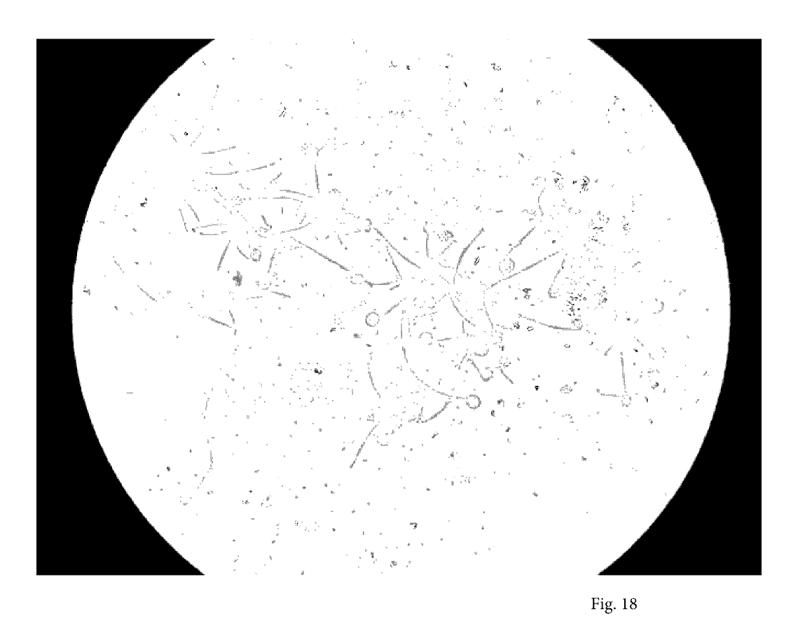

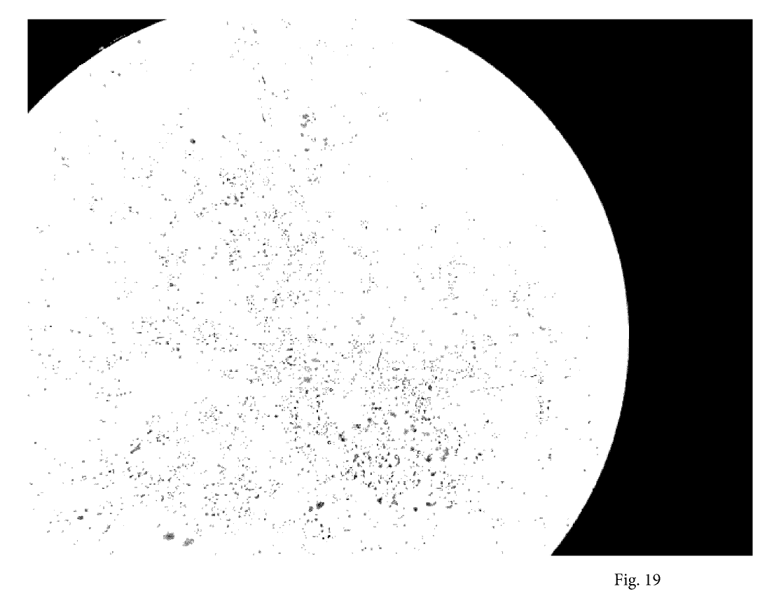

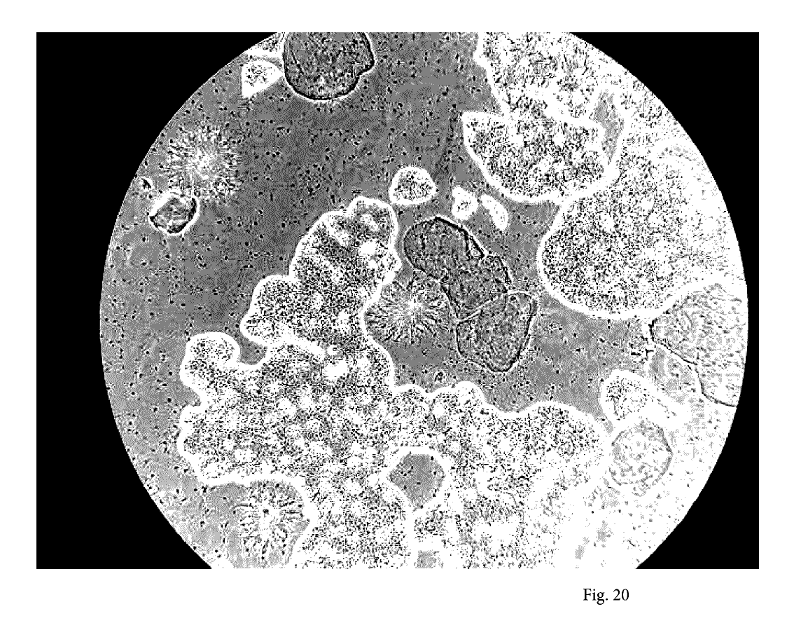

Referring now to FIGS. 4-21, when pathogens such as Borrelia sp., protozoa (including Babesia sp. & FL1953), and other biofilm-forming microbes, are observed in accordance with the present invention, the activity and progression of the various microorganisms cultured in a tissue sample is illustrated. For example, if Borrelia is contained within a tissue sample, the first observation is often the time when Borrelia can be observed in several forms. Some Borrelia can be seen balled up within encapsulations, often called cysts. Other Borrelia can be observed in the process of emerging from cysts, or in a free-form state with no encapsulation. Sometimes the Borrelia can be seen moving very fast across the field of view. As each microbe, including various forms, has its own distinct hue, shade, coloration, luminosity, color value, or saturation under phase contrast microscopy, the difference in color is part of what allows the scientist to determine what they are seeing.

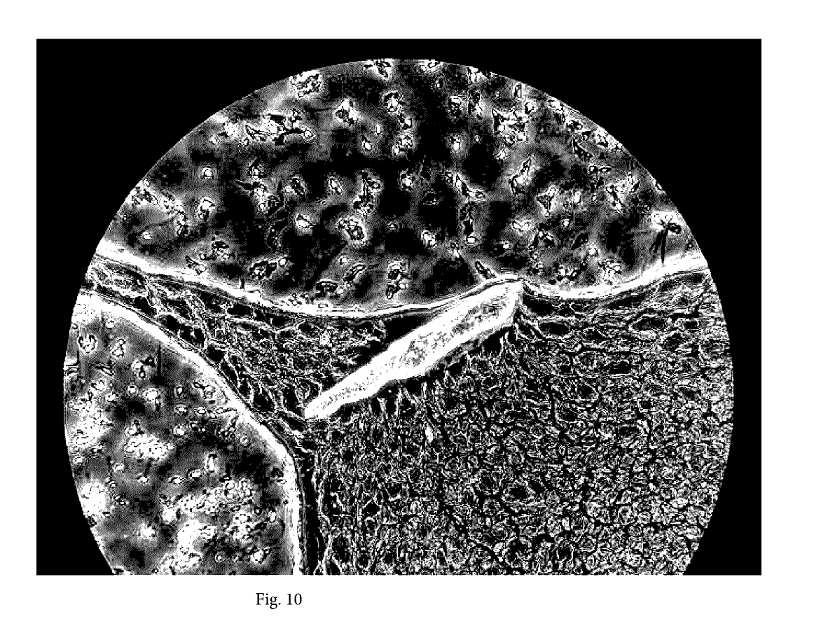

With respect to FIGS. 9 and 10, it is believed that there is no other organism besides FL1953 that produces structures like the biofilm columns illustrated therein.

As morphology is the other main defining characteristic, it is vital that the live Borrelia can be observed for several days in this culture, while the moisture slowly evaporates. Sometimes after a day or more into the culturing period, large groups of Borrelia can be observed surrounding a clump of what appears to be biofilm. But once the culture dries up completely, the Borrelia will no longer change form, and the culturing ends.

It is contemplated that while saliva is exemplified above as the source of human tissue sample, the process of preparing a microbe culture in accordance with the present invention may be employed with samples from any bodily fluids and tissues, such as saliva, blood, spinal fluid, and skin.

Furthermore, while Borrelia sp. and Babesia have been cited herein, the method of observing live microorganisms in human tissue samples may be employed to identify and observe a variety of microorganisms, including protozoa (including but not limited to malaria and amoeba), bacteria, and fungi.

It is appreciated that the instant method can be used to detect and treat rheumatological/autoimmune diseases such as multiple sclerosis (“MS”), lupus, amyotrophic lateral sclerosis (“ALS”), diabetes, and so forth, as well as diseases whose cause may be not fully understood. Indeed, it is believed that as Borrelia can cause what we think of as autoimmune disorders (such as MS, lupus, ALS, diabetes), the instant method can be employed to detect and direct treatment for these diseases.

It is understood that while human tissue is specifically referenced, the instant invention may be practiced with tissue samples from other animals.

The present invention has been shown and described herein in what is considered to be the most practical and preferred embodiment. It is recognized, however, that departures may be made therefrom within the scope of the invention and that obvious modifications will occur to a person skilled in the art.

What is claimed is:

1. A method of observing live microorganisms in an animal tissue sample over an extended duration of time which enables the detection and identification of multiple different microbes present in the sample, comprising the steps of:
preparing a sample containing animal tissue for viewing, wherein the step of preparing includes at least positioning the sample on a transparent specimen viewing member in a manner which allows the sample to remain a viable environmental analog after the step of preparing is completed;
performing by a phase contrast microscope an initial observation of the sample on the transparent specimen viewing member; and
after at least one selected time interval which allows objects in the sample to naturally undergo changes in morphology, performing by the phase contrast microscope at least one subsequent observation of the sample on the transparent specimen viewing member so as to facilitate analysis of the activity of selected objects in the sample.

2. The method of claim 1, wherein the phase contrast microscope utilizes at least one light emitting diode for any requisite illumination.

3. The method of claim 1, wherein the sample defines a portion of animal tissue which has been hydrated prior to being positioned on the transparent specimen viewing member.

4. The method of claim 1, wherein the step of preparing additionally includes positioning a transparent cover on the sample.

5. The method of claim 4, wherein the transparent cover defines a cover slip.

6. The method of claim 1, wherein the transparent specimen viewing member defines a microscope slide.

7. The method of claim 1, wherein positioning the sample on the transparent specimen viewing member in a manner which allows the sample to remain a viable environmental analog after the step of preparing is completed is defined by placing the sample on the transparent specimen viewing member and placing a transparent cover directly on top of the sample with no additional pressure or movement placed on the sample by the transparent cover.

8. The method of claim 1, additionally comprising a step of capturing by a discrete camera having a camera lens with a lower diameter than an eyepiece lens on the phase contrast microscope at least one of a still image and a video image.

9. A method of observing live microorganisms in an animal tissue sample over an extended duration of time which enables the detection and identification of multiple different microbes present in the sample, comprising the steps of:
preparing a sample containing animal tissue for viewing, wherein the step of preparing includes at least positioning the sample on a transparent specimen viewing member in a manner which allows the sample to remain a viable environmental analog after the step of preparing is completed by placing the sample on the transparent specimen viewing member and placing a transparent cover directly on top of the sample with no additional pressure or movement placed on the sample by the transparent cover;
performing by a phase contrast microscope an initial observation of the sample on the transparent specimen viewing member, wherein the phase contrast microscope utilizes at least one light emitting diode for any requisite illumination; and
after at least one selected time interval which allows objects in the sample to naturally undergo changes in morphology, performing by the phase contrast microscope at least one subsequent observation of the sample on the transparent specimen viewing member so as to facilitate analysis of the activity of selected objects in the sample.

10. The method of claim 9, wherein the sample defines a portion of animal tissue which has been hydrated prior to being positioning on the transparent specimen viewing member; and

11. The method of claim 10, wherein the transparent cover defines a cover slip.

12. The method of claim 10, wherein the transparent specimen viewing member defines a microscope slide.

13. The method of claim 10, additionally comprising a step of capturing by a discrete camera having a camera lens with a lower diameter than an eyepiece lens on the phase contrast microscope at least one of a still image and a video image.

14. A method of observing live microorganisms in an animal tissue sample over an extended duration of time which enables the detection and identification of multiple different microbes present in the sample, comprising the steps of:
configuring a phase contrast microscope to at least one of resolve features at between 200×-4000× in a phase contrast setting and resolve features below 350 nm in a phase contrast setting;
preparing a sample containing animal tissue for viewing, wherein the step of preparing includes at least positioning the sample on a transparent specimen viewing member and positioning a transparent cover on the sample in a manner which allows the sample to remain a viable environmental analog after the step of preparing is completed; and
performing by a phase contrast microscope an initial observation of the sample on the transparent specimen viewing member.

15. The method of claim 14, wherein the sample defines a portion of animal tissue which has been hydrated prior to being positioning on the transparent specimen viewing member.

16. The method of claim 14, wherein positioning the sample on the transparent specimen viewing member in a manner which allows the sample to remain a viable environmental analog after the step of preparing is completed is defined by placing the sample on the transparent specimen viewing member and placing a transparent cover directly on top of the sample with no additional pressure or movement placed on the sample by the transparent cover.

17. The method of claim 16, additionally comprising a step of capturing by a discrete camera having a camera lens with a lower diameter than an eyepiece lens on the phase contrast microscope at least one of a still image and a video image.

18. The method of claim 17, wherein the phase contrast microscope utilizes at least one light emitting diode for any requisite illumination.

19. The method of claim 14, wherein the phase contrast microscope utilizes at least one light emitting diode for any requisite illumination.

20. The method of claim 14, additionally comprising a step of capturing by a discrete camera having a camera lens with a lower diameter than an eyepiece lens on the phase contrast microscope at least one of a still image and a video image.
